(19) United States
(12) Patent Application Publication
(10) Pub. No.: US 2026/0224857 A1
(43) Pub. Date: Aug. 6, 2026

(54) COIL BODY AND MEDICAL DEVICE

(71) Applicant: ASAHI INTECC CO., LTD., Seto-shi (JP)

(72) Inventors: Kazuharu YOSHII, Aichi (JP); Kenta TSUGE, Aichi (JP); Hiromu OZAKI, Aichi (JP)

(73) Assignee: ASAHI INTECC CO., LTD., Seto-shi (JP)

(21) Appl. No.: 19/531,056

(22) Filed: Feb. 5, 2026

(30) Foreign Application Priority Data
Feb. 5, 2025 (JP) 2025-017240

(51) Int. Cl. A61M 25/09

(52) U.S. Cl. CPC A61M 25/09; A61M 2025/09083

(57) ABSTRACT

A coil body includes one or more strands, the one or more strands including a first strand portion and a second strand portion, the second strand portion including an outer winding part provided outside an outer periphery of the first strand portion and an inner winding part provided inside the outer periphery of the first strand portion, in a radial direction of the coil body.

### CROSS-REFERENCE TO RELATED APPLICATIONS

This application claims priority from Japanese Patent Application No. 2025-017240 filed February 5, 2025. The entire content of the priority application is incorporated herein by reference.

### TECHNICAL FIELD

The present disclosure relates to a coil body and a medical device.

### BACKGROUND

Japanese Patent Application No. H10-328309 discloses a guidewire with a small-diameter wire coil and a large-diameter wire coil.

### SUMMARY

A coil body provided in a guidewire contributes to torquability to a distal end side of the guidewire. In guidewires, there is a need to improve torquability from a proximal end side to a distal end side.

This specification discloses a technology capable of improving torquability.

A coil body according to an aspect is a coil body including one or more strands helically wound, the one or more strands including a first strand portion and a second strand portion, the second strand portion including an outer winding part provided outside an outer periphery of the first strand portion and an inner winding part provided inside the outer periphery of the first strand portion, in a radial direction of the coil body.

### BRIEF DESCRIPTION OF THE DRAWINGS

FIG. 1 is a schematic view of a guidewire according to a first embodiment.

FIG. 2 is a partial cross-sectional view of the guidewire according to the first embodiment.

FIG. 3 is a schematic view of a one-pitch range of the guidewire projected to a transverse cross section according to the first embodiment.

FIG. 4 is a schematic view of a guidewire according to a second embodiment.

FIG. 5 is a schematic view of a one-pitch range of the guidewire projected to a transverse cross section according to the second embodiment.

FIG. 6 is a schematic view of a guidewire according to a third embodiment.

FIG. 7 is a schematic view of a guidewire according to a fourth embodiment.

FIG. 8 is a schematic view of a one-pitch range of a guidewire according to a fifth embodiment projected to a transverse cross section.

FIG. 9 is a schematic view of a guidewire according to a sixth embodiment.

FIG. 10 is a schematic view of a one-pitch range of the guidewire projected to a transverse cross section according to the sixth embodiment.

FIG. 11 is a schematic view of a guidewire according to a seventh embodiment.

### DETAILED DESCRIPTION

Guidewires according to some embodiments will be described with reference to the drawings. The present disclosure is not limited to the embodiments described in the drawings. The guidewires shown in each drawing are shown in one example dimension to facilitate understanding of the implementation, and the actual dimensions are not limited to this.

In this specification, “distal end side” and “distal end direction” mean the side and direction in which a distal tip of a guidewire is located. “Proximal end side” and “proximal end direction” mean the opposite side and direction of “distal end side” and “distal end direction”. The “distal end” refers to an end portion on the distal end side of an arbitrary portion, and the “proximal end” refers to an end portion on the proximal end side of an arbitrary portion.

### First Embodiment

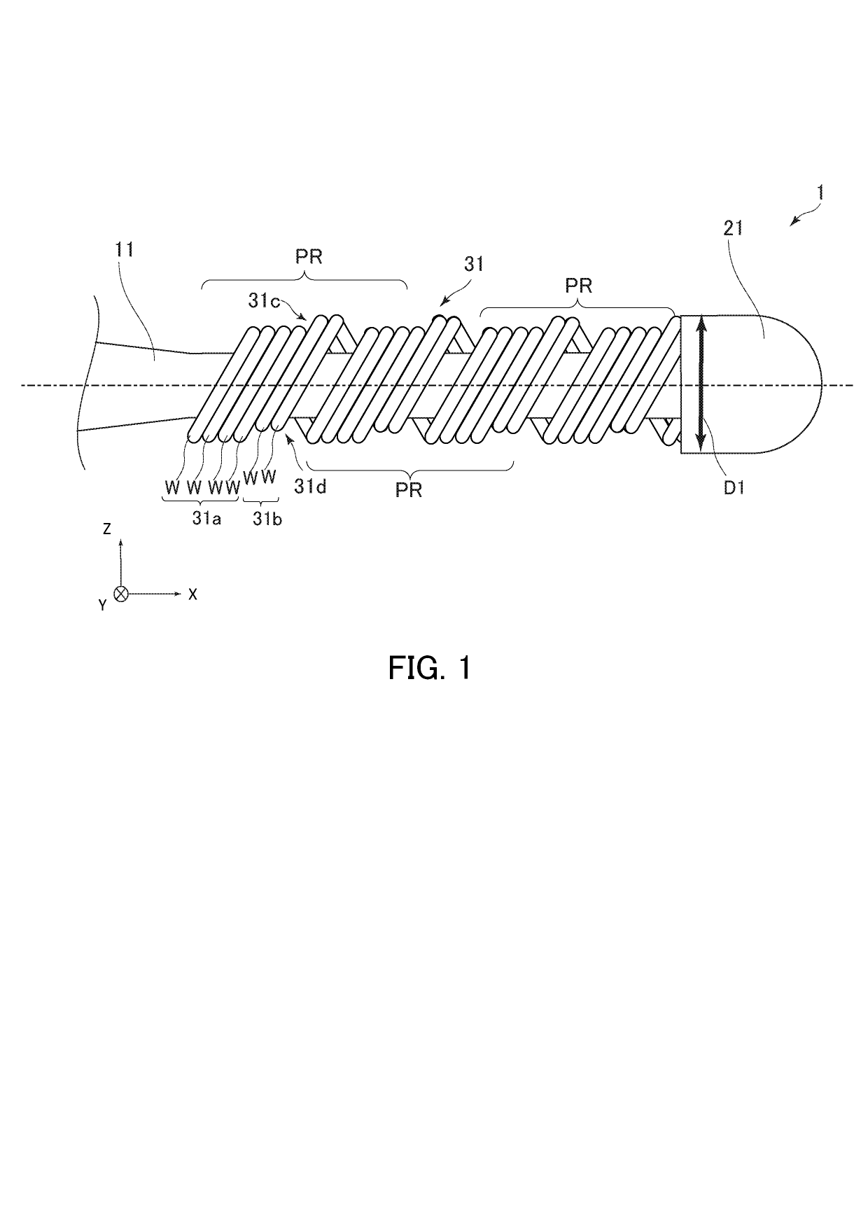

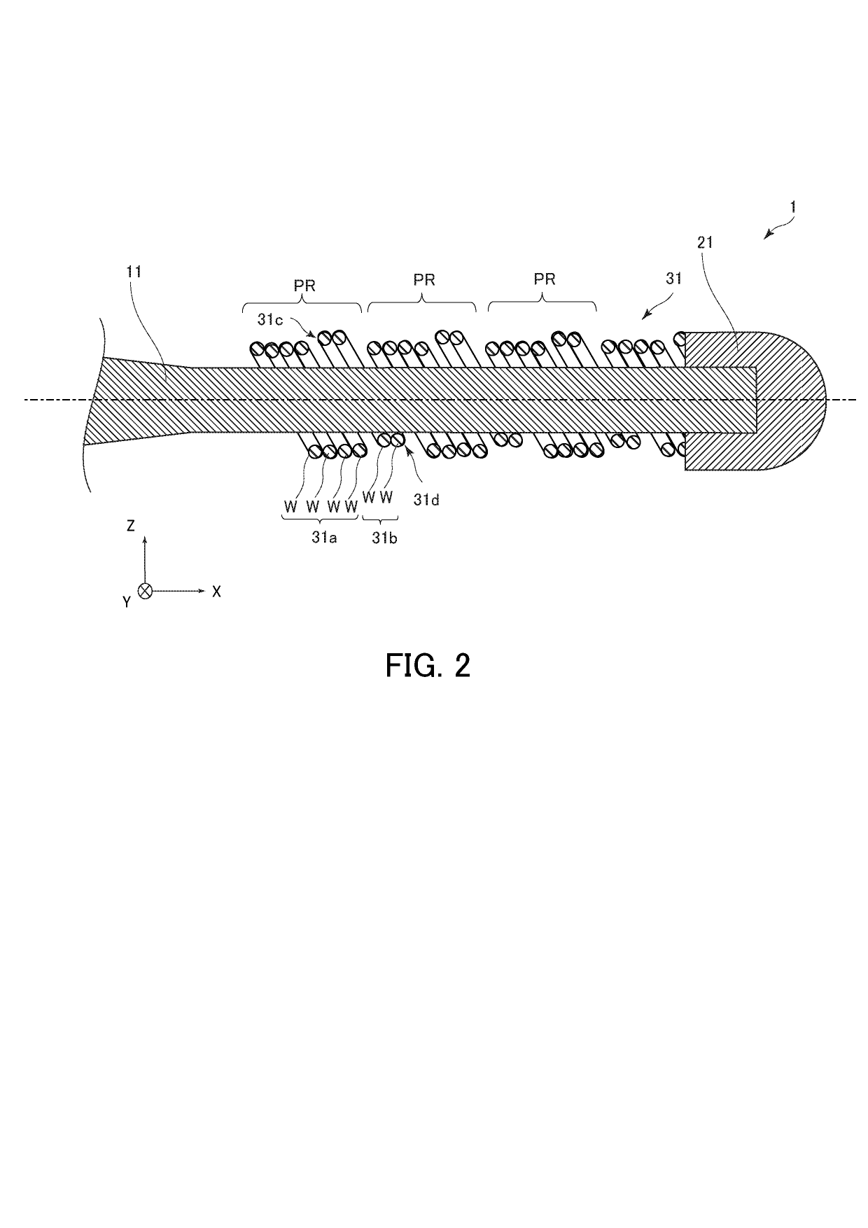

FIG. 1 is a schematic view of a guidewire according to a first embodiment. FIG. 2 is a partial cross-sectional view of the guidewire according to the first embodiment. FIG. 2 is a cross-sectional view of the guidewire cut along a plane including a center axis of the guidewire.

A guidewire 1 includes a core shaft 11, a distal tip 21, and a coil body 31.

The core shaft 11 is a long shaft extending from a proximal end to a distal end of the guidewire 1. In the present embodiment, the core shaft 11 has a substantially cylindrical shape and has a constant outer diameter at the distal end side. The distal end of the core shaft 11 is connected to the distal tip 21.

The material of the core shaft 11 may be, for example, stainless steel such as SUS304, Ni-Ti alloy, Co-Cr alloy, or the like. The total length of the core shaft 11 may be, for example, 1,800 to 3,000 mm.

The distal tip 21 has, for example, a convex shape such as a hemispherical shape, toward the distal end side, and is provided at the distal end of the guidewire 1. The distal tip 21 joins the distal end of the core shaft 11 to the distal end of the coil body 31. The material of the distal tip 21 may be, for example, a brazing material such as aluminum alloy solder, silver solder, or gold solder, metal solder such as Ag-Sn alloy or Au-Sn alloy, or adhesive such as epoxy-based adhesive.

The distal tip 21 is provided with the axis of the core shaft 11 as a center. A diameter D1 of the distal tip 21 in the radial direction of the guidewire 1 is large enough to encompass the range of existence of the coil body 31 when the coil body 31 is projected to the transverse cross section of the guidewire 1, that is, to the Z-Y plane. Specifically, the diameter D1 of the distal tip 21 in the radial direction of the guidewire

1 is equal to or more than twice the distance from the center of the core shaft 11 to the most radially distant position of the second strand portion 31b of the coil body 31, which is described below.

The coil body 31 is provided so as to cover the outer periphery of the distal end side of the core shaft 11. The coil body 31 is a hollow cylindrical coil with a plurality of strands W helically wound around the core shaft 11. In the examples shown in FIGS. 1 and 2, the coil body 31 includes six strands W as a unit helically wound multiple times. In the present embodiment, the diameters of the strands W are the same.

The strands W may be one or more solid wires, or one or more twisted wires. The material of strands W may be, for example, stainless steel such as SUS316, superelastic alloys such as Ni-Ti alloys, or radiopaque metals such as platinum or tungsten. The cross-sectional shape of the strands W is circular in the example in FIG. 2. The cross-sectional shape of the strands W may be elliptical, triangular, rectangular, square, or the like.

The coil body 31 includes a first strand portion 31a with four strands W on the proximal end side and a second strand portion 31b with two strands W on the distal end side in a one-pitch range PR with one winding of six strands. The state in which the strands W are wound in the first strand portion 31a is different from the state in which the strands W are wound in the second strand portion 31b. Specifically, the second strand portion 31b includes an outer winding part 31c provided outside the outer periphery of the first strand portion 31a and an inner winding part 31d provided inside the outer periphery of the first strand portion 31a in the radial direction of the coil body 31. In the outer periphery of the coil body 31, the portion corresponding to the outer winding part 31c of the second strand portion 31b is convex and the portion corresponding to the inner winding part 31d of the second strand portion 31b is concave.

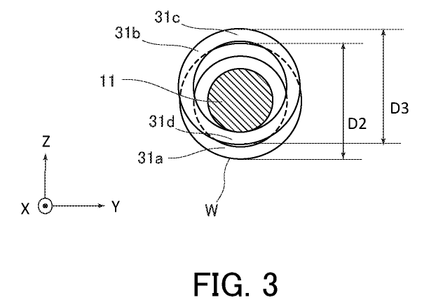

FIG. 3 is a schematic view of a one-pitch range of the guidewire projected to a transverse cross section according to the first embodiment.

The range where the strands W of the second strand portion 31b exist is displaced in a positive direction of a Z-axis relative to the range where the strands W of the first strand portion 31a exist. In other words, the center of the second strand portion 31b when the strands W are wound and the center of the first strand portion 31a when the strands W are wound are displaced from each other. The outer diameter D2 of the first strand portion 31a and the outer diameter D3 of the second strand portion 31b have the same size. The second strand portion 31b includes an outer winding part 31c located on a radially outward side from the first strand portion 31a and an inner winding part 31d located on a radially inward side from the first strand portion 31a. The inner peripheral side of the inner winding part 31d may be in contact with the core shaft 11.

According to this guidewire, the inner peripheral side of the inner winding part 31d of the second strand portion 31b is more likely to interfere with the core shaft 11. This interference results in efficient torque transmission between the core shaft 11 and the coil body 31, which can improve torquability to the distal end in the guidewire 1.

### Second Embodiment

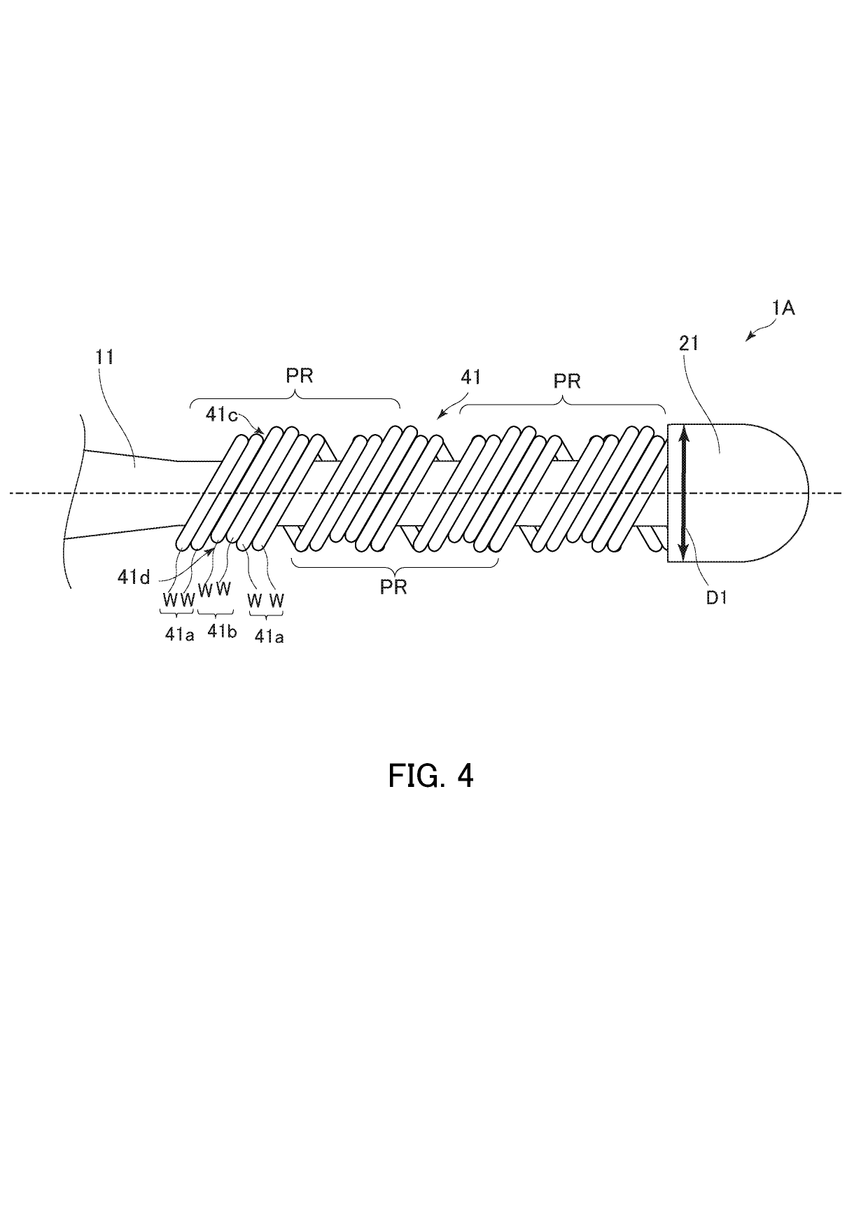

FIG. 4 is a schematic view of a guidewire according to a second embodiment. In a guidewire 1A according to the second embodiment, parts similar to those of the guidewire 1 of the first embodiment will be denoted by the same symbols and an explanation thereof may be omitted.

The guidewire 1A includes a coil body 41 instead of the coil body 31 of the guidewire 1.

The coil body 41 includes six strands W as a unit helically wound multiple times.

The coil body 41 includes a first strand portion 41a with two strands W on the proximal end side and two strands W on the distal end side and a second strand portion 41b with two strands W in the middle in a one-pitch range PR with one winding of six strands. The state in which the strands W are wound in the first strand portion 41a is different from the state in which the strands W are wound in the second strand portion 41b. Specifically, the second strand portion 41b includes an outer winding part 41c provided outside the outer periphery of the first strand portion 41a and an inner winding part 41d provided inside the outer periphery of the first strand portion 41a in the radial direction of the coil body 41. In the outer periphery of the coil body 41, the portion corresponding to the outer winding part 41c of the second strand portion 41b is convex and the portion corresponding to the inner winding part 41d of the second strand portion 41b is concave.

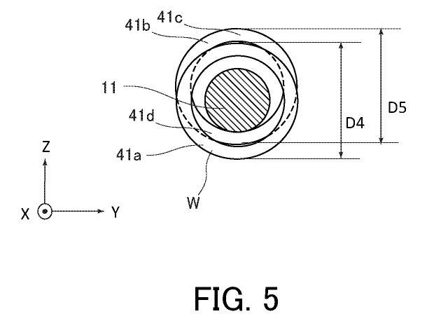

FIG. 5 is a schematic view of a one-pitch range of the guidewire projected to a transverse cross section according to the second embodiment.

The range where the strands W of the second strand portion 41b exist is displaced in a positive direction of the Z-axis relative to the range where the strands W of the first strand portion 41a exist. The outer diameter D4 of the first strand portion 41a and the outer diameter D5 of the second strand portion 41b are the same. The second strand portion 41b includes an outer winding part 41c located on a radially outward side from the first strand portion 41a and an inner winding part 41d located on a radially inward side from the first strand portion 41a. The inner peripheral side of the inner winding part 41d may be in contact with the core shaft 11.

According to this guidewire, the inner peripheral side of the inner winding part 41d of the second strand portion 41b is more likely to interfere with the core shaft 11. This interference results in efficient torque transmission between the core shaft 11 and the coil body 41, which can improve torquability to the distal end in the guidewire 1.

### Third Embodiment

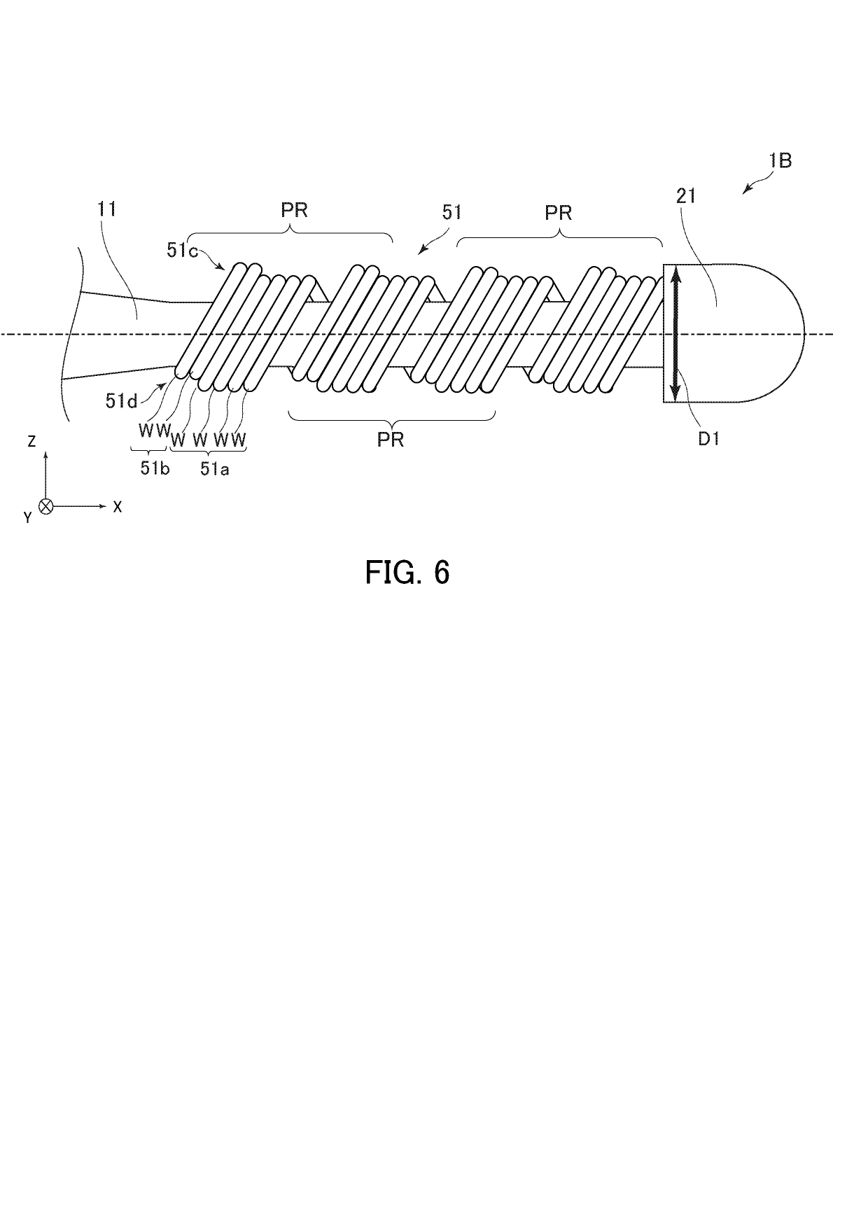

FIG. 6 is a schematic view of a guidewire according to a third embodiment. In a guidewire 1B according to the third embodiment, parts similar to those of the guidewire 1 of the first embodiment will be denoted by the same symbols and an explanation thereof may be omitted.

The guidewire 1B includes a coil body 51 instead of the coil body 31 of the guidewire 1.

The coil body 51 includes six strands W as a unit helically wound multiple times.

The coil body 51 includes a first strand portion 51a with four strands W on the distal end side and a second strand portion 51b with two strands W on the proximal end side in a one-pitch range PR with one winding of six strands. The state in which the strands W are wound in the first strand portion 51a is different from the state in which the strands W are wound in the second strand portion 51b. Specifically, the second strand portion 51b includes an outer winding part 51c provided outside the outer periphery of the first strand portion 51a and an inner winding part 51d provided inside the outer periphery of the first strand portion 51a in the radial direction of the coil body 51. In the outer periphery of the coil body 51, the portion corresponding to the outer winding part 51c of the second strand portion 51b is convex and the portion corresponding to the inner winding part 51d of the second strand portion 51b is concave.

According to this guidewire, the inner peripheral side of the inner winding part 51d of the second strand portion 51b is more likely to interfere with the core shaft 11. This interference results in efficient torque transmission between the core shaft 11 and the coil body 51, which can improve torquability to the distal end in the guidewire 1.

### Fourth Embodiment

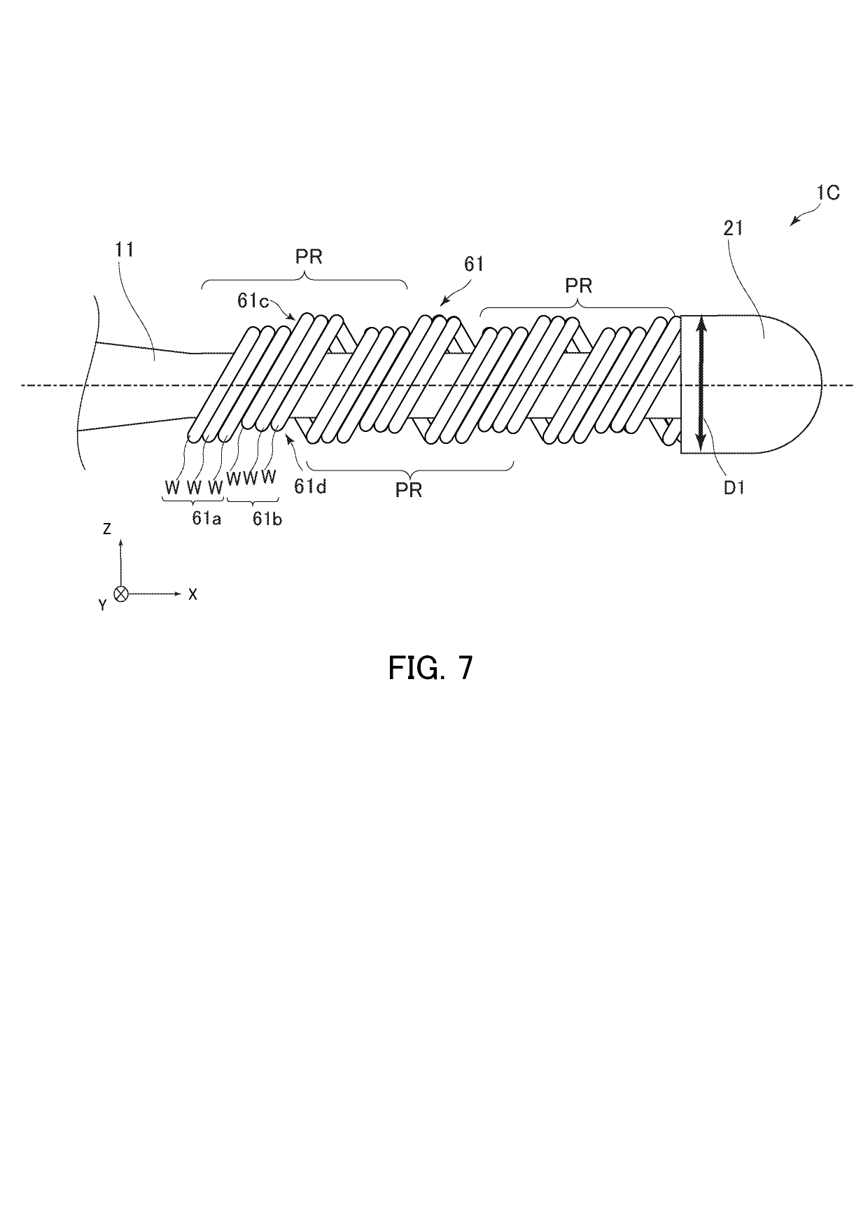

FIG. 7 is a schematic view of a guidewire according to a fourth embodiment. In a guidewire 1C according to the fourth embodiment, parts similar to those of the guidewire 1 of the first embodiment will be denoted by the same symbols and an explanation thereof may be omitted.

The guidewire 1C includes a coil body 61 instead of the coil body 31 of the guidewire 1.

The coil body 61 includes six strands W as a unit helically wound multiple times.

The coil body 61 includes a first strand portion 61a with three strands W on the proximal end side and a second strand portion 61b with the same number of strands W as that of the first strand portion 61a in a one-pitch range PR with one winding of six strands. The state in which the strands W are wound in the first strand portion 61a is different from the state in which the strands W are wound in the second strand portion 61b. Specifically, the second strand portion 61b includes an outer winding part 61c provided outside the outer periphery of the first strand portion 61a and an inner winding part 61d provided inside the outer periphery of the first strand portion 61a in the radial direction of the coil body 61. In the outer periphery of the coil body 61, the portion corresponding to the outer winding part 61c of the second strand portion 61b is convex and the portion corresponding to the inner winding part 61d of the second strand portion 61b is concave.

According to this guidewire, the inner peripheral side of the inner winding part 61d of the second strand portion 61b is more likely to interfere with the core shaft 11. This interference results in efficient torque transmission between the core shaft 11 and the coil body 61, which can improve torquability to the distal end of the guidewire 1.

### Fifth Embodiment

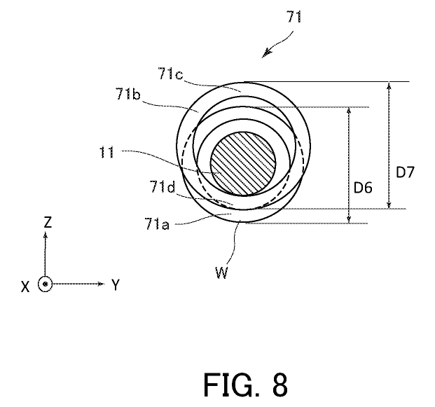

FIG. 8 is a schematic view of a one-pitch range of a guidewire projected to a transverse cross section according to a fifth embodiment. In the guidewire according to the fifth embodiment, parts similar to those of the guidewire 1 of the first embodiment will be denoted by the same symbols and an explanation thereof may be omitted.

The guidewire according to the fifth embodiment includes a coil body 71 instead of the coil body 31 of the guidewire 1.

In the coil body 71, the second strand portion 31b by the two strands W on the distal end side of the coil body 71 is substituted with a second strand portion 71b. A first strand portion 71a is similar to the first strand portion 31a.

The range where the strands W of the second strand portion 71b exist is displaced in a positive direction of the Z-axis relative to the range where the strands W of the first strand portion 71a exist. The outer diameter D6 of the first strand portion 71a and the outer diameter D7 of the second strand portion 71b are larger than the outer diameter D6 of the first strand portion 71a. The second strand portion 71b includes an outer winding part 71c located on a radially outward side from the first strand portion 71a and an inner winding part 71d located on a radially inward side from the first strand portion 71a. The inner peripheral side of the inner winding part 71d may be in contact with the core shaft 11.

According to this guidewire, the inner peripheral side of the inner winding part 71d of the second strand portion 71b is more likely to interfere with the core shaft 11. This interference results in efficient torque transmission between the core shaft 11 and the coil body 71, which can improve torquability to the distal end of the guidewire 1.

### Sixth Embodiment

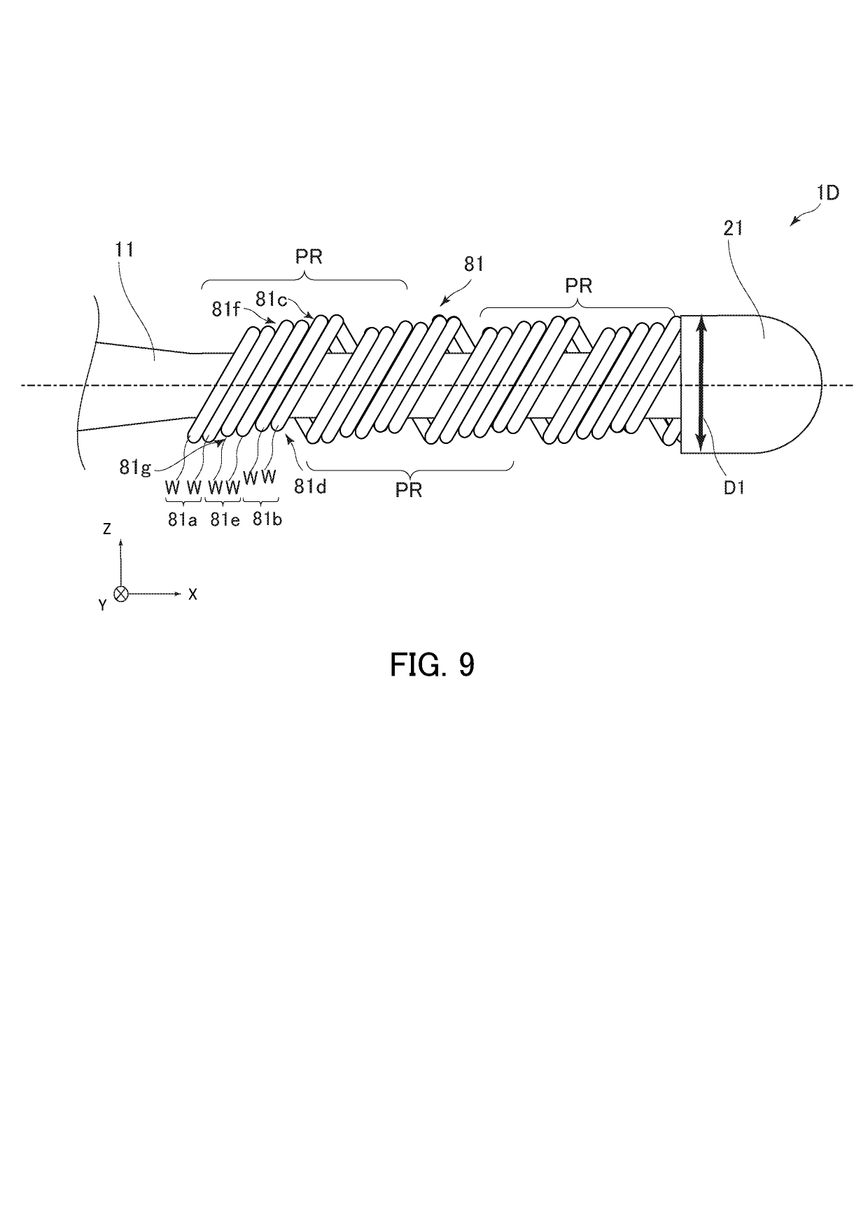

FIG. 9 is a schematic view of a guidewire according to a sixth embodiment. In a guidewire 1D according to the sixth embodiment, parts similar to those of the guidewire 1 of the first embodiment will be denoted by the same symbols and an explanation thereof may be omitted.

The guidewire 1D includes a coil body 81 instead of the coil body 31 of the guidewire 1.

The coil body 81 includes six strands W as a unit helically wound multiple times.

The coil body 81 includes a first strand portion 81a with two strands W on the proximal end side, a second strand portion 81b with two strands W on the distal end side, and a third strand portion 81e with two strands W in the middle, in a one-pitch range PR with one winding of six strands. The state in which the strands W are wound in the first strand portion 81a, the state in which the strands W are wound in the second strand portion 81b, and the state in which the strands W are wound in the third strand portion 81e are different. Specifically, the second strand portion 81b includes an outer winding part 81c provided outside the outer periphery of the first strand portion 81a and an inner winding part 81d provided inside the outer periphery of the first strand portion 81a in the radial direction of the coil body 81. The third strand portion 81e includes a first intermediate winding portion 81f provided outside the outer periphery of the first strand portion 81a and inside the outer winding part 81c, and a second intermediate winding part 81g provided inside the outer periphery of the first strand portion 81a and outside the inner winding part 81d, in the radial direction of the coil body 81.

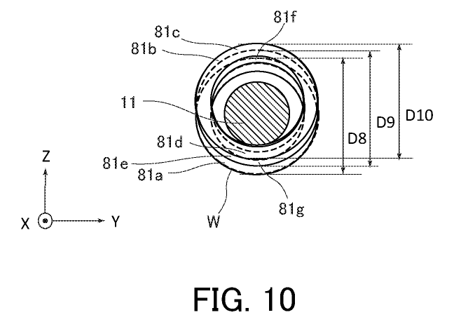

FIG. 10 is a schematic view of a one-pitch range of the guidewire projected to a transverse cross section according to the sixth embodiment.

The range where the strands W of the second strand portion 81b exist is displaced in a positive direction of the Z-axis relative to the range where the strands W of the first strand portion 81a exist. The range where the strands W of the third strand portion 81e exist is located in the middle in the Z-axis direction between the range where the strands W of the first strand portion 81a exist and the range where the strands W of the second strand portion 81b exist. The outer diameter D8 of the first strand portion 81a, the outer diameter D10 of the second strand portion 81b, and the outer diameter D9 of the third strand portion 81e are the same. The second strand portion 81b includes an outer winding part 81c located on a radially outward side from the first strand portion 81a and an inner winding part 81d located on a radially inward side from the first strand portion 81a. The inner peripheral side of the inner winding part 81d may be in contact with the core shaft 11. The third strand portion 81e includes a first intermediate winding part 81f located on a radially outward side from the first strand portion 81a and on a radially inward side from the outer winding part 81c, and a second intermediate winding part 81g located on a radially inward side from the first strand part 81a and on a radially inward side from the inner winding part 81d.

According to this guidewire, the inner peripheral side of the inner winding part 81d of the second strand portion 81b is more likely to interfere with the core shaft 11. This interference results in efficient torque transmission between the core shaft 11 and the coil body 81, which can improve torquability to the distal end of the guidewire 1.

### Seventh Embodiment

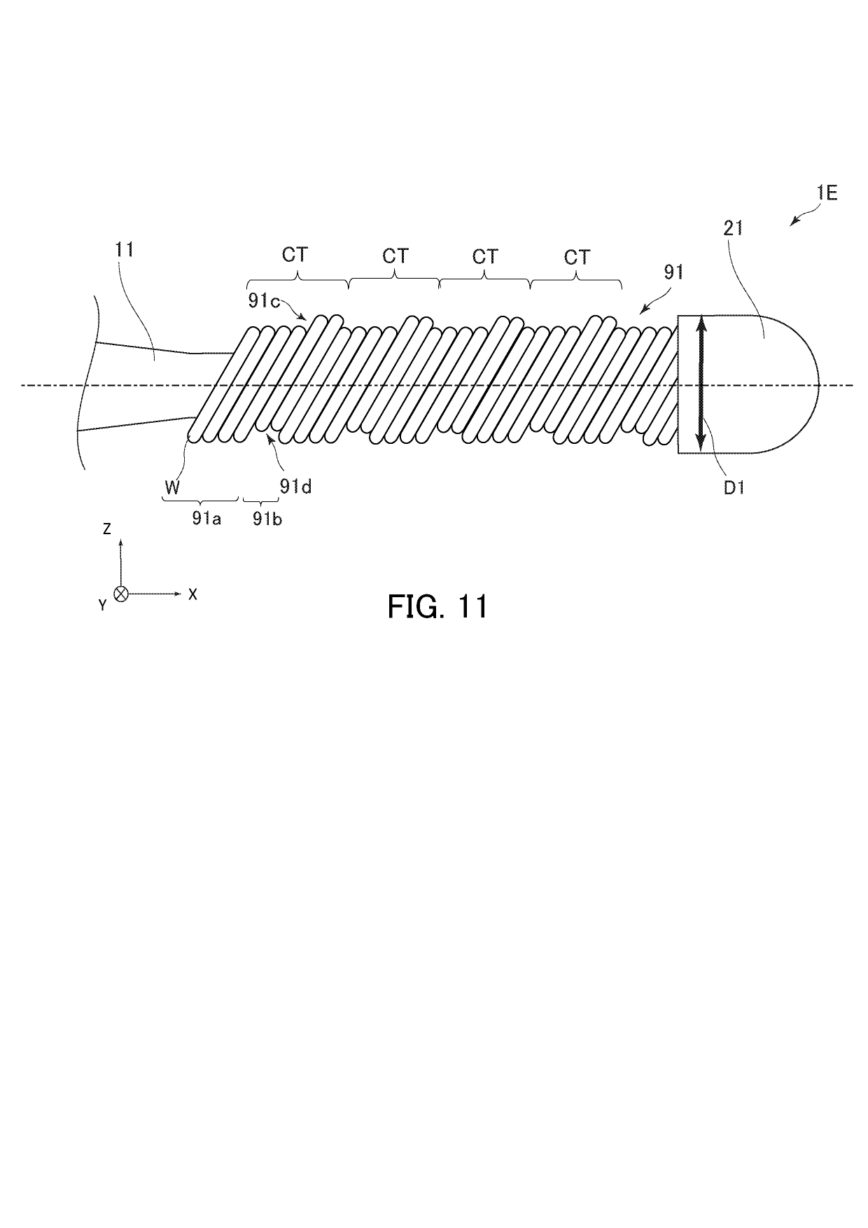

FIG. 11 is a schematic view of a guidewire according to a seventh embodiment. In a guidewire 1E according to the seventh embodiment, parts similar to those of the guidewire 1 of the first embodiment will be denoted by the same symbols and an explanation thereof may be omitted.

The guidewire 1E includes a coil body 91 instead of the coil body 31 of the guidewire 1.

The coil body 91 includes one strand W helically wound multiple times.

The coil body 91 includes a first strand portion 91a including four windings of the strand W on the proximal end side and a second strand portion 91b including two windings of the strand W on the distal end side in a predetermined winding range CT with six windings of the strand W. The state in which the strand W is wound in the first strand portion 91a is different from the state in which the strand W is wound in the second strand portion 91b. Specifically, the second strand portion 91b includes an outer winding part 91c provided outside the outer periphery of the first strand portion 91a and an inner winding part 91d provided inside the outer periphery of the first strand portion 91a in the radial direction of the coil body 91. In the outer periphery of the coil body 91, the portion corresponding to the outer winding part 91c of the second strand portion 91b is convex and the portion corresponding to the inner winding part 91d of the second strand portion 91b is concave.

According to this guidewire, the inner peripheral side of the inner winding part 91d of the second strand portion 91b is more likely to interfere with the core shaft 11. This interference results in efficient torque transmission between the core shaft 11 and the coil body 91, which can improve torquability to the distal end of the guidewire 1.

The technology disclosed in this specification is not limited to the above-described embodiments and modifications, and can be modified in various forms without departing from the gist thereof. For example, the following modifications are also possible.

In the embodiments described above, the second strand portion is provided in each one-pitch range in the coil body. Not limited to this, the second strand portion may be provided only in some one-pitch ranges of the coil body.

In the above embodiments, the strands of the second strand portion in each one-pitch range in the coil body are the same strands. Not limited to this, the strands of the second strand portion in a one-pitch range may be different from the strands of the second strand portion in another one-pitch range.

In the above embodiments, the diameters of the strands W are the same. Not limited to this, the diameters of some of the strands W may be different.

In the above embodiments, the proximal end side of the coil body may be joined to the core shaft.

In the above embodiments, the guidewire has been described as a medical device including a coil body. Not limited to this, the coil body may be provided in a medical device other than the guidewire.

The disclosure is not limited to the configuration of the above embodiments, but is defined by the terms of the claims and is intended to include any modifications within the scope and meaning equivalent to the terms of the claims.

The disclosure includes the following supplementary notes.

### Supplementary note 1

A coil body (31, 41, 51, 61, 71, 81, 91) including one or more strands (W) helically wound, in which

the one or more strands (W) include a first strand portion (31a, 41a, 51a, 61a, 71a, 81a, 91a) and a second strand portion (31b, 41b, 51b, 61b, 71b, 81b, 91b), and

the second strand portion (31b, 41b, 51b, 61b, 71b, 81b, 91b) includes an outer winding part (31c, 41c, 51c, 61c, 71c, 81c, 91c) provided outside an outer periphery of the first strand portion (31a, 41a, 51a, 61a, 71a, 81a, 91a) and an inner winding part (31d, 41d, 51d, 61d, 71d, 81d, 91d) provided inside the outer periphery of the first strand portion, in a radial direction of the coil body.

### Supplementary note 2

The coil body (31, 41, 51, 61, 71, 81) according to supplementary note 1, including a plurality of the strands (W), in which the plurality of strands (W) include the first strand portion (31a, 41a, 51a, 61a, 71a, 81a) and the second strand portion (31b, 41b, 51b, 61b, 71b, 81b) within a one-pitch range (PR) being a range of one winding of the plurality of strands.

### Supplementary note 3

The coil body (31, 41, 51, 61, 71, 81) according to supplementary note 2, in which an outer periphery of the coil body is formed with a protruding portion by the outer winding part (31c, 41c, 51c, 61c, 71c, 81c) and a concave portion by the inner winding part (31d, 41d, 51d, 61c, 71c, 81c).

### Supplementary note 4

The coil body (31, 41, 51, 61, 71, 81) according to supplementary note 2 or 3, in which an external shape of the first strand portion (31a, 41a, 51a, 61a, 71a, 81a) and an external shape of the second strand portion (31b, 41b, 51b, 61b, 71b, 81b) projected to a transverse cross section of the coil body are circular, and a diameter of strands of the first strand portion (31a, 41a, 51a, 61a, 71a, 81 a) and a diameter of strands of the second strand portion (31b, 41b, 51b, 61b, 71b, 81b) are the same.

### Supplementary note 5

The coil body (31, 41, 51, 71) according to any one of supplementary notes 2 to 4, in which the number of strands (W) of the first strand portion (31a, 41a, 51a, 71a) is different from the number of strands (W) of the second strand portion (31b, 41b, 51b, 71b).

### Supplementary note 6

The coil body (61) according to any one of supplementary notes 2 to 5, in which the number of strands (W) of the first strand portion (61a) is the same as the number of strands (W) of the second strand portion (61b).

### Supplementary note 7

The coil body (31, 51) according to supplementary note 5, in which the number of strands (W) of the second strand portion (31b, 51b) is less than the number of strands (W) of the first strand portion (31a, 51a), and the second strand portion (31b, 51b) is located at an end within the one-pitch range (PR).

### Supplementary note 8

The coil body (41) according to supplementary note 5, in which the number of strands (W) of the second strand portion (41b) is less than the number of strands (W) of the first strand portion (41a), and the second strand portion (41b) is located in a middle within the one-pitch range (PR).

### Supplementary note 9

The coil body (31, 41, 51, 61, 71, 81) according to any one of supplementary notes 2 to 8, including a plurality of one-pitch ranges (PR), in which, within the plurality of one-pitch ranges, the strands (W) of the first strand portion (31a, 41a, 51a, 61a, 71a, 81a) are the same strands (W) and the strands (W) of the second strand portion (31b, 41b, 51b, 61b, 71b, 81b) are the same strands (W).

### Supplementary note 10

The coil body (81) according to any one of supplementary notes 2 to 9, including the first strand portion (81a), the second strand portion (81b), and a third strand portion (81e) within the one-pitch range (PR), in which the third strand portion (81e) include: a first intermediate winding part (81f) provided outside the outer periphery of the first strand portion (81a) and inside the outer winding part (81c), and a second intermediate winding part (81g) provided inside the outer periphery of the first strand portion (81a) and outside the inner winding part (81d), in a radial direction of the coil body (81).

### Supplementary note 11

A medical device (1, 1A, 1B, 1C, 1D, 1E) including a core shaft (11) and a coil body (31, 41, 51, 61, 71, 81, 91) covering an outer periphery of the core shaft (11), in which

the coil body (31, 41, 51, 61, 71, 81, 91) is the coil body (31, 41, 51, 61, 71, 81, 91) according to any one of supplementary notes 1 to 10.

1. A coil body comprising one or more strands helically wound, wherein
the one or more strands include a first strand portion and a second strand portion, and
the second strand portion includes an outer winding part provided outside an outer periphery of the first strand portion and an inner winding part provided inside the outer periphery of the first strand portion, in a radial direction of the coil body.

2. The coil body according to claim 1, comprising a plurality of the strands, wherein the plurality of strands include a first strand portion and a second strand portion within a one-pitch range being a range of one winding of the plurality of strands.

3. The coil body according to claim 2, wherein an outer periphery of the coil body is formed with a protruding portion by the outer winding part and a concave portion by the inner winding part.

4. The coil body according to claim 2, wherein an external shape of the first strand portion and an external shape of the second strand portion projected to a transverse cross section of the coil body are circular, and a diameter of strands of the first strand portion and a diameter of strands of the second strand portion are the same.

5. The coil body according to claim 2, wherein a number of strands of the first strand portion is different from a number of strands of the second strand portion.

6. The coil body according to claim 2, wherein a number of strands of the first strand portion is the same as a number of strands of the second strand portion.

7. The coil body according to claim 5, wherein the number of strands of the second strand portion is less than the number of strands of the first strand portion, and the second strand portion is located at an end within the one-pitch range.

8. The coil body according to claim 5, wherein the number of strands of the second strand portion is less than the number of strands of the first strand portion, and the second strand portion is located in a middle within the one-pitch range.

9. The coil body according to claim 2, comprising a plurality of one-pitch ranges, wherein, within the plurality of one-pitch ranges, the strands of the first strand portion are the same strands and the strands of the second strand portion are the same strands.

10. The coil body according to claim 2, comprising the first strand portion, the second strand portion, and a third strand portion within the one-pitch range, wherein the third strand portion includes a first intermediate winding part provided outside the outer periphery of the first strand portion and inside the outer winding part, and a second intermediate winding part provided inside the outer periphery of the first strand portion and outside the inner winding part, in a radial direction of the coil body.

11. A medical device comprising a core shaft and a coil body covering an outer periphery of the core shaft, wherein
the coil body is the coil body according to claim 1.
